(19) United States
(12) Patent Application Publication
(10) Pub. No.: US 2025/0119074 A1
(43) Pub. Date: Apr. 10, 2025

(54) ENERGY HARVESTER BASED ON PIEZOELECTRIC IMPACT COMPARTMENT

(71) Applicants: AALBORG UNIVERSITET, Aalborg Øst (DK); REGION NORDJYLLAND, Aalborg Øst (DK)

(72) Inventors: Alireza REZANIAKOLAEI, Aalborg Ø (DK); Sam RIAHI, Aalborg (DK); Ali Asghar ENKESHAFI, Aalborg SV (DK); Majid KHAZAEE, Aalborg Ø (DK); Lasse Aistrup ROSENDAHL, Aalborg SØ (DK)

(21) Appl. No.: 18/730,000

(22) PCT Filed: Jan. 19, 2023
(86) PCT No.: PCT/DK2023/050010

§ 371 Date: Jul. 18, 2024

(30) Foreign Application Priority Data
Jan. 24, 2022 (DK) PA 2022 70029

(51) Int. Cl. H02N 2/18; A61N 1/375; A61N 1/378

(52) U.S. Cl. CPC H02N 2/183; A61N 1/37512; A61N 1/3785

(57) ABSTRACT

Disclosed is an energy harvester for generating electric energy from an external vibratory force, such as a vibratory force caused by movement of a part of a living human or animal. The energy harvester has a compartment (CMP1, CMP2) with wall element(s) forming a cavity inside. At least one wall element of the compartment has a piezoelectric element connected to an electric output terminal. An impact object (BL) is positioned inside the cavity of the compartment. The impact object is configured to move inside the cavity and to generate an impact on a surface of the wall element(s), so as to cause the piezo-electric element to generate an electric output on the electric output terminal. The compartment may have a cubic shape, e.g. made of a single piezoelectric element folded into shape, e.g. a two-layer piezoelectric element. The impact object may be a metal or ceramics object with a spherical shape. Especially, the energy harvester may be used to power an implantable medical device, e.g. an intra-cardiac pacemaker. An energy harvester housing within an intra-cardiac pacemaker has been tested to be sufficient for powering the pacemaker stimulation circuit by the vibratory force caused by heartbeats.

### FIELD OF THE INVENTION

The present invention relates to the field energy harvesters, more specifically for piezoelectric energy harvesters. More specifically, the invention provides an energy harvester based on a piezoelectric impact compartment. Even more specifically, the invention relates to self-powering implantable devices, such as intra-cardiac pacemakers.

### BACKGROUND OF THE INVENTION

Implantable medical device, such as pacemaker, such as intra-cardiac pacemakers, require electric power for functioning. Normally, a battery delivers the electric power to such devices, but batteries have a limited lifetime and thus a surgical procedure is required for replacement. For example, a cardiac pacemaker can have a battery lifetime of such as 10-15 years.

When battery replacement is required, a risk, discomfort and substantial costs are involved in the surgical procedure.

Even further, long-life batteries for such implantable devices occupy a significant space of the implantable device.

Various solutions for self-powering devices are known. However, it is a problem to harvest enough electric energy within the small dimension of a compact medical device, such as a leadless intra-cardiac pacemaker.

### SUMMARY OF THE INVENTION

Thus, according to the above description, it is an object of the present invention to provide a compact and yet effective energy harvester, in particular for powering implantable devices, such as intra-cardiac pacemakers.

In a first aspect, the invention provides an energy harvester for generating electric energy from an external vibratory force, such as a vibratory force caused by movement of a part of a living human or animal, the energy harvester comprising
at least one compartment with one or more wall elements forming a cavity inside, wherein one or more of the wall elements comprises at least one piezoelectric element connected to an electric output terminal, and
at least one impact object positioned inside the cavity in each of the at least one compartments, wherein the at least one impact object is configured to move inside the cavity and to generate an impact on a surface of the one or more wall elements when the at least one compartment is excited by the external vibratory force, so as to cause the at least one piezoelectric element to generate an electric output on the electric output terminal.

Such energy harvester is advantageous, since it has been found that it is possible to generate a significant amount of electric energy by impact energy provided by the piezoelectric compartment and impact object even at small dimensions. Only a small and cheap rechargeable battery is required compared to the large and expensive long-life batteries used in prior art devices.

Especially, it has been found possible to generate electric energy for powering an intra-cardiac pacemaker by means of the vibratory force of heartbeats within the casing of the intra-cardiac pacemaker, i.e. within a cylindrical shape with a length smaller than 30 mm and with a diameter smaller than 10 mm. With an energy harvester with two cubic shaped compartments and spherical impact objects inside, it has been found that the energy harvester can generate multiple electric output pulses per heartbeat which is sufficient for powering the pacemaker stimulation circuit.

The compartment and the impact object therein can have various sizes and shapes, e.g. the shape of the compartment and the impact object is preferably designed to match the expected type of external vibratory force for optimal energy harvesting. Especially, it has been found preferable to design the compartment and impact object so match the external vibratory force, as to generate multiple electric pulses for each vibratory cycle, in case the vibratory force is a cyclic force.

It is to be understood that the energy harvester can also be used in other types of devices than miniature implantable medical devices, e.g. devices with larger dimensions. Especially, the energy harvester can be formed by one compartment with an impact object inside or by a plurality of sets of compartments each with an impact object inside.

The energy harvester is further advantageous in that it can be implemented by rather simple components. Furthermore, it can be manufactured in rather simple manufacturing processes, and therefore, the energy harvester is suited for low cost mass production.

In the following, preferred features and embodiments of the energy harvester will be mentioned.

### Compartment Shape Details

Preferably, the cavity inside the compartment forms a free space in which the impact object can move freely, caused by the vibrational force acting on the compartment. Thus, preferably the impact object has no structural connection to the compartment inhibiting its movements, but rather the impact object can move freely inside the cavity and thus impact with any surface of wall element(s) forming the cavity, only determined by the vibrational force on the compartment.

The energy harvester may comprise a compartment wherein the one or more wall elements form a cavity with an inner surface having a curved part. Especially, the one or more wall elements form a cavity with a concave inner surface, such as a spherical or elliptical inner surface.

Especially, it may be preferred that the compartment has a cavity which has an ellipsoidal shape, specifically having a cavity with an ellipsoidal inner surface. Specifically, the compartment may be formed by one single piezoelectric element shaped to provide the ellipsoidal shape.

Especially, the one or more wall elements may form a cavity with an inner surface having a plurality of curved parts, such as a plurality of curved parts with different curved shapes.

Especially, the energy harvester may comprise a compartment wherein the one or more wall elements form a cavity with an inner surface comprising at least one plane part. More specifically, the one or more wall elements may form a cavity with an inner surface comprises a plurality of plane parts, e.g. forming a polygonal shape, such as 2-20 plane parts, such as 3-10 plane parts, such as 4-8 plane parts. More specifically, the one or more wall elements may form a cavity with an inner surface comprising 6 plane parts, such as the one or more wall elements forming a cubic shaped cavity.

The energy harvester may comprise a compartment wherein the one or more wall elements form a cavity with an inner surface comprising a plurality of differently shaped parts. Especially, the one or more wall elements form a cavity with an inner surface comprising a plurality of curved parts and a plurality of plane parts.

The one or more wall element(s) may form an enclosure around the at least one impact object, wherein the enclosure has a maximum opening size which is smaller than a size of the at least one object. Hereby, the impact object will not fall out of the compartment by accident. Especially, the enclosure may be made water or even airtight. However, the compartment may alternatively be formed with wall element(s) forming a cage around the impact object, thus with a plurality of openings shaped so in relation to the size of the impact object, that the impact object cannot escape from the compartment.

### Compartment Implementation Details

The energy harvester may comprise a plurality of compartments, such as 2-10 compartments, e.g. a comprising a plurality of differently shaped compartments or a plurality of compartments with identical shapes, such as comprising at least two compartments with a cubic shape. Specifically, at least two of the plurality of compartments with identical shapes may be mechanically connected so that corresponding inner surfaces of their cavities have different angles, thereby allowing a higher probability of harvesting energy with a random external vibratory force pattern. The electric output terminals from the plurality of compartments may be interconnected, such as via respective electric circuits, to form one common electric output.

The energy harvester may have a compartment with cavity having a volume of 1-1000 mm³, such as 1-500 mm³, such as 10-500 mm³, such as 10-200 mm³.

The energy harvester may have a compartment with more than 20% of an inner surface area of the cavity is formed by one or more wall elements comprising a piezoelectric element. Especially, more than 50% of an inner surface area of the cavity, such as 50-100% of an inner surface area, such as 90-100% of an inner surface area, of an inner surface area of the cavity may be formed by one or more wall elements comprising a piezoelectric element. It may be preferred that a high fraction, such as all of the inner surface of the cavity is covered by wall elements having a surface made of a piezoelectric material, or at least is in mechanical connection so as to ensure that impact force from the impact object with wall element is transmitted to an piezoelectric element, thereby contributing to generating electric energy.

The energy harvester may have one wall element comprising at least one piezoelectric element which is bent or folded so as to form a plurality of different inner surface parts of the cavity. Specifically, one wall element may comprise one single piezoelectric element which is bent or folded so as to form the compartment, such as a cubic compartment. With one single piezoelectric element forming the entire inner surface of the compartment cavity, all impact energy is captured by this piezoelectric element, thus generating an electric output at one single electric terminal. This is advantageous with respect to simplicity of the following electric circuits, e.g. an AC to DC converter, since only one converter circuit is required per compartment.

In some embodiments, the compartment may alternatively be formed with a cavity having an ellipsoidal shape, e.g. manufactured as one single piezoelectric element forming the entire inner surface of the compartment cavity, thus with the above-mentioned advantage of simplicity, and with the additional advantage of increased durability due to the smooth inner surface of the compartment cavity.

This is possible to do in a manufacturing process. However, a compartment with a cavity having an ellipsoidal shape may alternatively be made of two or more separate piezoelectric parts attached together to form the ellipsoidal shaped cavity.

The energy harvester may have at least one wall element which is made of a piezoelectric sandwich structure. Especially, such sandwich structure may comprise two piezoelectric layers, such as each piezoelectric layer having a thickness of 10-1000 μm, such as 50-500 μm. Especially, all wall elements forming the cavity may be made of a piezoelectric sandwich structure, such as a sandwich structure comprising two piezoelectric layers, such as each piezoelectric layer having a thickness of 10-1000 μm, such as 50-500 μm, e.g. with an intermediate layer of non-piezoelectric material having a thickness of 10-1000 μm. Such sandwich structure has been found to be suitable, e.g. for bending into the shape of a preferred compartment shape, such that the entire compartment is made of one piezoelectric sandwich element.

### Impact Object Details

The energy harvester may have a spherically shaped impact object. Alternatively, the impact object may be or have a polygonal shape, such as a hexagonal shape.

The impact object may be made of a metal or a ceramic, which has been found as a suitable material to provide an efficient impact with the inner surface of the wall element(s). Specifically, the impact object may be spherically shaped and made of metal or ceramics.

If preferred, a plurality of impact objects may be positioned inside one compartment. Specifically, 2-4, 2-10 or even more than 10 separate impact objects may be positioned inside the compartment.

In one embodiment, at least one compartment has an impact object positioned inside which is a spherical object, such as a metal or ceramics object, wherein the impact object has a diameter of 1-5 mm and a mass of 0.05-3 g. More specifically, the at least one compartment may have a cubic shape with a side length being within 2-10 mm, such as the cubic compartment being formed by one single piezoelectric element bent into a cubic shape, e.g. a piezoelectric element being a sandwich structure comprising two piezoelectric sheets and an intermediate layer of non-piezoelectric material. Such embodiment has been found to provide a high electric efficiency in spite of the compact dimension as is thus suited for implantable medical devices, e.g. intra-cardiac pacemakers.

Preferably, at least one compartment is formed by one single piezoelectric element. E.g. the piezoelectric element can be a sheet of piezoelectric material which is bent into the shape, e.g. a cubic shape, of the compartment. This allows a rather simple manufacturing process and all electric energy harvested by the compartment is provided at one single electric terminal, thereby providing a simple interface to further electric circuits, converters, energy storages etc. More specifically, two or more compartments may be formed by respective single piezoelectric elements.

In some embodiments, at least one compartment is formed by two or more separate piezoelectric elements with respective electric output terminals.

In some embodiments, at least one compartment may have an impact object positioned inside which has a volume of 1-80% of a volume of the cavity inside the compartment. Specifically, the impact object may have a volume of such as 1-60%, such as 1-40%, such as 10-80%, such as 20-60%, such as 30-50% of a volume of the cavity inside the compartment.

In some embodiments, at least one compartment has an impact object positioned inside which has a volume of 2-40%, such as 2-30%, such as 3-20%, such as 5-15%, of a volume of the cavity inside the compartment.

Preferably, the impact object positioned inside the compartment has a mass and size optimized for optimal electric energy harvesting based on knowledge about the external vibratory force.

In some embodiments, the energy harvester comprises an electric circuit, such as a circuit comprising an AC to DC converter, connected to the electric output terminal of the piezoelectric material to receive electric energy from the piezoelectric material. An AC to DC converter has been found to provide a more effective harvesting of the electric pulses generated by the piezoelectric material. The energy harvester may further comprise an electric storage, e.g. comprising a rechargeable battery or a capacitor or the like, so as to generate a stable DC voltage for powering an electric consuming device.

In a second aspect, the invention provides a device comprising at least one energy harvester according to the first aspect.

The device may comprise an electric storage, and wherein the energy harvester is connected to provide electric energy to the electric storage. The electric storage may comprise a rechargeable battery, a capacitor or the like.

The device may comprise an AC to DC converter with an electric input connected to the electric output terminal of the energy harvester to receive electric energy from the energy harvester and to generate a DC electric output accordingly. Specifically, the AC to DC converter may be connected with its DC electric output to an electric storage and/or to an electric consuming device.

The device may comprise an electric consuming device, wherein the energy harvester is connected to power the electric consuming device. The energy harvester may be directly connected to power the electric consuming device or indirectly via an intermediate electric circuit, such as an AC to DC converter and/or an electric storage. Specifically, the energy harvester and the electric consuming device may be housed within one common casing. The electric consuming device may in principle be any electrically powered functional element that provides the primary or a secondary function of the device. Preferably, the energy harvester is designed to provide electric power for powering all of the electric consuming components of the device.

The device may comprise an energy harvester with a plurality of separate compartments, such as 2-10, each with respective impact objects inside.

In preferred embodiment, the device comprises a medical device, such as the device being a medical device. The medical device may be configured to provide a therapy on a living body of a human or animal, such as an electric or light stimulation or providing a substance such as a medicament to the living body. Especially, the medical device may be arranged for implantation in a body of a living human or animal.

In some embodiments, the device comprises a medical device being one of: a pacemaker, a defibrillator, a cardiac resynchronization therapy device, a nerve stimulation device, a pump device, a pain pacemaker, a bladder stimulator, and a cochlear implant.

In some embodiments, the device comprises a leadless intra-cardiac pacemaker, wherein the at least one energy harvester is configured to generate electric energy enough to power an electric heart stimulator circuit of the pacemaker. Specifically, the energy harvester, a re-chargeable battery, and the electric heart stimulator circuit are housed within one common casing. Specifically, the energy harvester has at least one compartment and an impact object configured to ensure a plurality of impacts between the impact object and the inner surface of the cavity of the compartment caused by the vibration force of one single heartbeat, when the intra-cardiac pacemaker is implanted for normal use inside a heart of a living human or animal.

In a third aspect, the invention provides a method for harvesting electric energy from an external vibratory force, such as a vibratory force caused by movement of a part of a living human or animal, the method comprising
providing a compartment with one or more wall elements forming a cavity inside, wherein at least one wall element comprises a piezoelectric material, and wherein an impact object is arranged inside the cavity,
vibrating the compartment by the external vibratory force so as to cause the impact object to move inside the cavity and to generate impacts on a surface of the at least one wall element comprising piezoelectric material, and
collecting electric energy from the piezoelectric material caused by said impacts.

The vibratory force may especially be caused by movement or movements of a part of a living human or animal. Specifically, the vibratory force is caused by heartbeats of a living human or animal.

In some embodiments, the method comprises attaching or fastening the compartment to a part of a living human or animal. Specifically, the method comprises attaching or fastening the compartment to a part inside a body of the living human or animal. Specifically, the method comprises attaching or fastening the compartment to a part outside a body of the living human or animal.

In preferred embodiments, the method comprises powering a medical device by the electric energy collected from the piezoelectric material. Specifically, the method comprises powering an intra-cardiac pacemaker by the electric energy collected from the piezoelectric material.

In some embodiments, the method comprises charging an electric storage based on the electric energy collected from the piezoelectric material. Specifically, this may involve charging a rechargeable battery.

In a fourth aspect, the invention provides a method of providing a therapy on a living human or animal body, such as providing electric stimulation to a part of the living human or animal body, based on electric energy from an energy harvester according to the first aspect.

In preferred embodiments, the method comprises providing an energy harvester according to the first aspect, mounting the energy harvester on or in a part of the living human or animal body, vibrating the energy harvester by a vibratory force provided by the living human or animal body. More specifically, the method may comprise collecting electric energy from the energy harvester and powering a therapy device, such as a pacemaker or another electrically powered therapy device, based on the collected electric energy. More specifically, the method comprises providing a therapy on the living human or animal body by means of the therapy device, e.g. providing electric stimulation on a part of the living human or animal body.

It to be understood that therapy may involve various types of interaction with the human or animal body. Including providing light and/or electric signals, e.g. to relieve pain on a limb or neck or back of the human or animal body. However, it may additionally or alternatively comprise providing a substance, e.g. a medicament to the human or animal body, the substance may be such as insulin or a pain relieving medicament or the like.

In a fifth aspect, the invention provides a method of providing a therapy on a living human or animal body, such as providing electric stimulation to a part of the living human or animal body, based on the device according to the second aspect.

It is appreciated that the described embodiments and features for the mentioned aspects can be intermixed in any way.

### BRIEF DESCRIPTION OF THE FIGURES

The invention will now be described in more detail with regard to the accompanying figures of which

FIG. 1 illustrates a simple block diagram of an energy harvester embodiment,

FIG. 2 illustrates sketches of possible shapes of the energy harvester compartment or of the impact object therein,

FIG. 3 illustrates a simple block diagram of a device embodiment, where the device is powered by the energy harvester of the invention,

FIG. 4 illustrates a simple sketch of a layout of components of an intra-cardiac pacemaker including the energy harvester of the invention,

FIG. 5a, 5B, 5c, and 5d illustrate sketches of components of an intra-cardiac pacemaker including various embodiments of the energy harvester of the invention,

FIG. 6 illustrates a sketch of a leadless intra-cardiac pacemaker with an energy harvester with two cubic compartments,

FIG. 7a illustrates three dimensional movement of a human or animal heart,

FIG. 7b illustrates position of an intra-cardiac pacemaker within the human or animal heart,

FIG. 8 illustrates a photo of an intra-cardiac pacemaker prototype with an energy harvesting embodiment of the invention which has been tested in an animal heart, and a cap of a pen to illustrate size,

FIG. 9-11 illustrate graphs indicating electrical measurement results of the tested energy harvesting embodiment,

FIG. 12a, 12b illustrate sketches of initial steps of a possible manufacturing of an energy harvesting compartment based on one single piece or sheet of piezoelectric material, and

FIGS. 13 and 14 illustrate steps of method embodiments.

The figures illustrate specific ways of implementing the present invention and are not to be construed as being limiting to other possible embodiments falling within the scope of the attached claim set.

### DETAILED DESCRIPTION OF THE INVENTION

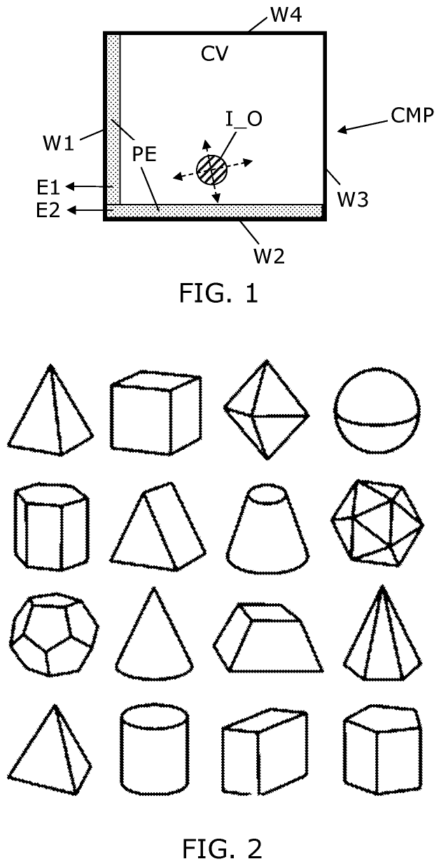

FIG. 1 illustrates a simple two dimensional block diagram of an energy harvester embodiment. A compartment CMP with wall elements W1-W4 forms a cavity CV inside. It is to be understood that the illustration is two dimensional for simplicity, but preferred embodiments have a three dimensional compartment CMP with a three dimensional cavity CV therein. The cavity CV can be free space, or if preferred filled with a fluid such as gas or liquid. The cavity CV can be a completely air or water tight enclosure or a more open structure.

In this embodiment, two wall elements W1, W2 have a piezoelectric element PE attached thereto, here shown on the inner surface of the wall elements W1, W2, i.e. the piezoelectric element PE covers a part of the inner surface of the cavity CV. The piezoelectric element PE is here shown connected to two electric output terminal E1, E2. However, if may be preferred that a plurality of the wall elements W1-W4 have one common piezoelectric element PE attached thereto, e.g. a piezoelectric element PE bent into the shape of the inner surface of the plurality of wall elements W1-W4, thereby requiring only one electric output terminal E1 from the common piezoelectric element PE. The piezoelectric element PE can in general be formed by any known type of piezoelectric material or layered structures comprising one or more layers of piezoelectric material(s).

At impact object I_O is positioned inside the cavity CV of the compartment CMP. The impact object I_O is configured to move inside the cavity CV and to generate impacts on surfaces of the cavity CV, when the compartment is excited by an external vibratory force. In this way, the piezoelectric element or elements PE will be hit by the mass of the impact object I_O, thereby causing the piezoelectric element PE to generate an electric pulse on the electric output terminal E1, E2. In this way, the energy harvester transfers an external vibratory force into electric energy.

Most preferably, the shape and size of the compartment CMP and the shape, size and mass of the impact object I_O are designed to match the expected external vibratory force, e.g. properties of the force such as amplitude and frequency content etc., so as to optimize energy harvesting. For cyclic vibratory forces, it is preferred that the compartment CMP and impact object I_O are designed such that multiple impacts, preferably several impacts, between the impact object I_O and surfaces of the cavity CV of the compartment CMP will occur during each vibration cycle. Hereby the most efficient energy harvesting can be obtained.

In a specific basic embodiment, the compartment CMP has a cubic shape, and the impact object I_O has a spherical shape and is made of metal or ceramics, e.g. a solid sphere or metal or ceramics.

The energy harvester has been tested to provide a high electric efficiency in spite of compact dimensions. It is to be understood that the energy harvester can in general be used for powering any type of electrically consuming devices based on any type of external vibratory force. The external vibratory force can be a vibrating machine or movements of a part of a living body of a human or animal.

Especially, it has been found advantageously for medical devices, such as implantable medical devices, and a prototype which can generate electric power enough by the vibration caused by heartbeats to power the stimulation electronics of a leadless intra-cardiac pacemaker, and still the energy harvester can be housed inside the casing with the compact dimensions acceptable for that type of pacemaker. The prototype has been tested in a controlled experiment implanted in the heart of a pig.

FIG. 2 illustrates sketches of possible shapes of the energy harvester compartment or of the impact object therein. Apart from basic rectangular box or cubic shapes, various polygonal shapes are seen, e.g. hexagonal based shapes, which may be preferred in some cases. Further, spherical or ellipsoid shapes may be preferred for the impact object but may also be used for the compartment. Further, various tubular or cylindrical based shapes as well a cone based shapes may also be preferred in some cases. It is to be understood that the size of the cavity inside the compartment is larger than the size of the impact object, since the impact object should be able to move inside the cavity in order to generate impact energy.

It is to be understood that the compartment cavity as well as the impact object may have any shape, the possibilities shown in FIG. 2 serving only as examples.

The preferred shape depends of the space available for the energy harvester, and the type of external vibratory force expected to be applied to the compartment of the energy harvester. In case of a cyclic vibratory force, it is preferred that the compartment is shaped such that it is ensured, that the impact object will impact the surface of the cavity of the compartment multiple times per vibratory cycle.

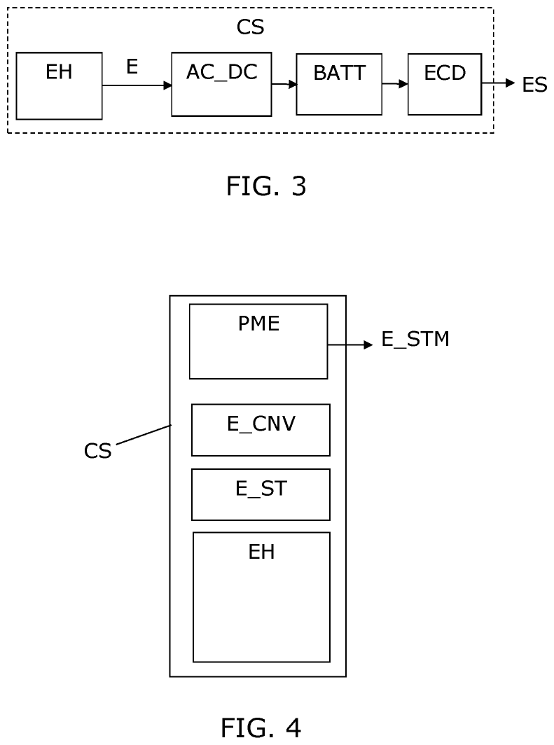

FIG. 3 illustrates a simple block diagram of a device embodiment comprising the energy harvester EH according to the invention. The energy harvester EH generates electric impulses E which are converted to a DC voltage by means of an AC to DC converter. The DC voltage is applied to an energy storage BATT, e.g. comprising a rechargeable battery, which in turn is used to power an electrically consuming device ECD. All components EH, AC_DC, BATT, ECD are shown housed inside one common casing CS. In this specific embodiment the electrically consuming device ECD comprises an electric stimulation circuit which generates an electric stimulation signal ES to be applied externally via an electrode placed outside the casing CS. Specifically, the device may be a medical device, or more specifically an intra-cardiac pacemaker or a pain relieving device. The casing CS may be suited for attaching to a living body or suited for implanting in the living body by an interventional method.

FIG. 4 illustrates a simple sketch of a layout of components of a leadless intra-cardiac pacemaker including the energy harvester EH of the invention. The energy harvester EH is shown located at one end of an elongated, e.g. cylindrical, casing CS. Pacemaker electronics PME, comprising an electric stimulation circuit, is located in the opposite end of the casing CS, and it is connected to an electrode which extends outside the casing CS so as to provide an electric stimulation E_STM to the heart tissue. An electric converter E_CNV and an electric storage E_ST are located in the casing CS between the energy harvester EH and the pacemaker electronics PME.

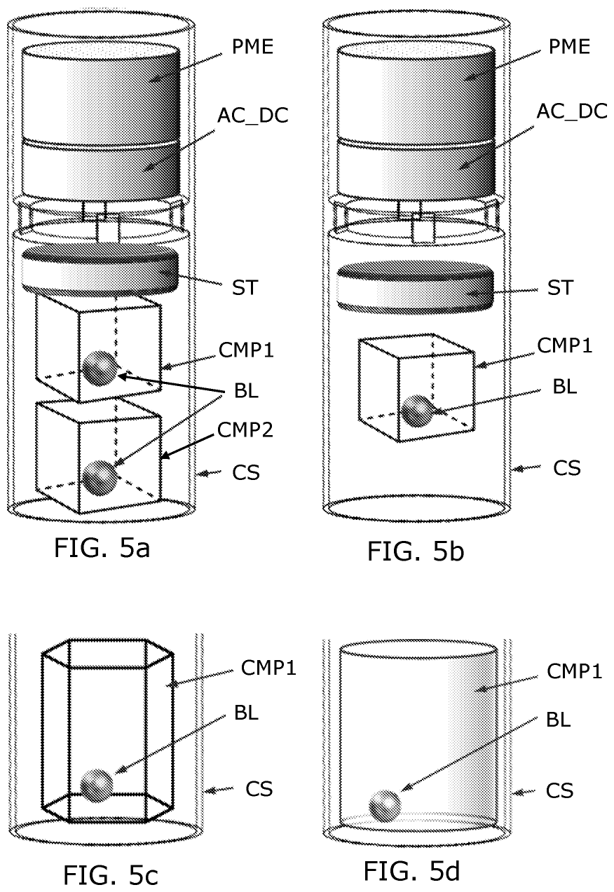

FIG. 5a, 5B, 5c, and 5d illustrate sketches of components of an intra-cardiac pacemaker including various embodiments of the energy harvester of the invention, i.e. more detailed sketches than shown in FIG. 4. All embodiments shown have an elongated casing CS, e.g. a cylindrical casing CS of a metal, suited for implanting in the heart of a living human or animal. In one end of the casing CS pacemaker electronics PME, an AC to DC electric converter, and an electric storage ST are located, while the energy harvester is shown located in the opposite end of the casing CS.

FIG. 5a illustrates an embodiment where the energy harvester comprises two cubically shaped compartments CMP1, CMP2 each with a spherical ball BL inside as impact object. The two compartments CMP1, CMP2 are located next to each other, e.g. adjacent to each other or slightly spaced apart, but the two compartments CMP1, CMP2 are preferably located at different longitudinal positions in the elongated casing CS.

The outer side lengths of the cubical compartments CMP1, CMP2 is preferably such as 3-8 mm, such as 4-6 mm, while the ball BL has a diameter of such as 2-4 mm and a mass of such as 0.05-0.2 grams.

FIG. 5b illustrates an embodiment where the energy harvester has only one single cubically shaped compartment CMP1 with a spherical ball shaped impact object BL.

FIG. 5c illustrates an embodiment where the energy harvester has a compartment CMP1 which has a hexagonal cross sectional area, i.e. the compartment CMP1 is formed by six plane wall elements in a hexagonal configuration and with a plane top and bottom thereby forming an enclosure inside the compartment CMP1. The impact object inside the compartment CMP1 is a spherical ball BL.

FIG. 5d show illustrates an embodiment where the energy harvester has a compartment CMP1 which has a cylindrical shape, i.e. having a curved sidewall and having a plane top and bottom. The impact object inside the compartment CMP1 is a spherical ball BL.

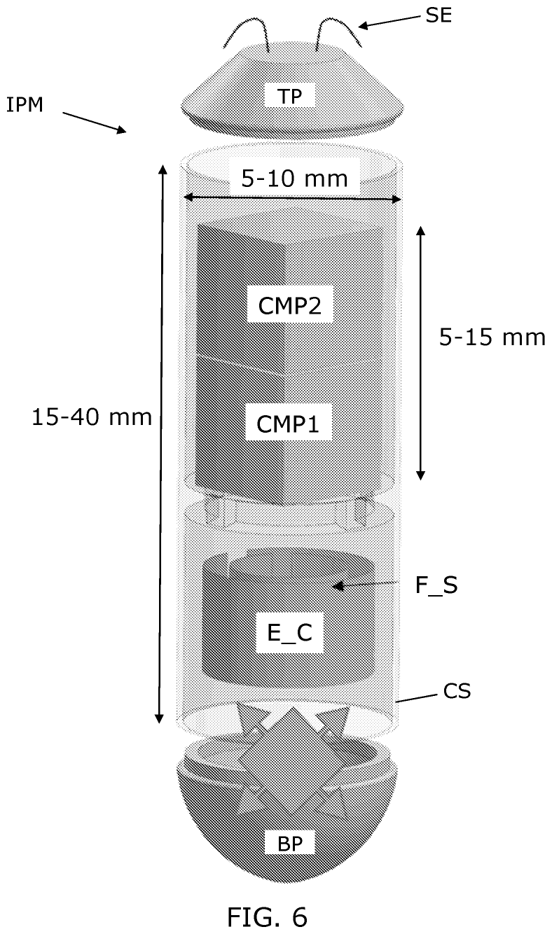

FIG. 6 illustrates a sketch of a leadless intra-cardiac pacemaker with an energy harvester with two cubic compartments CMP1, CMP2 each with side length of such as 3-6 mm. In this embodiment the two energy harvester compartments CMP1, CMP2 are stacked in an upper part of the cylindrical casing part CS, while an electronic circuit E_C is located in a lower part of the cylindrical casing part CS. The electronic circuit E_C is stimulation electrodes SE penetrating a top part TP of the casing connecting to the upper part of the cylindrical casing part CS, while a bottom part BP connects to a lower part of the cylindrical casing part CS so as to form a completely tight enclosure provided by the casing BP, CS, SE, when assembled for normal use.

The height of the cylindrical casing part CS may be such as 15-40 mm, while the width may be such as 5-10 mm. The energy harvester compartments CMP1, CMP2 may occupy such as 5-15 mm of a height of the cylindrical casing part CS.

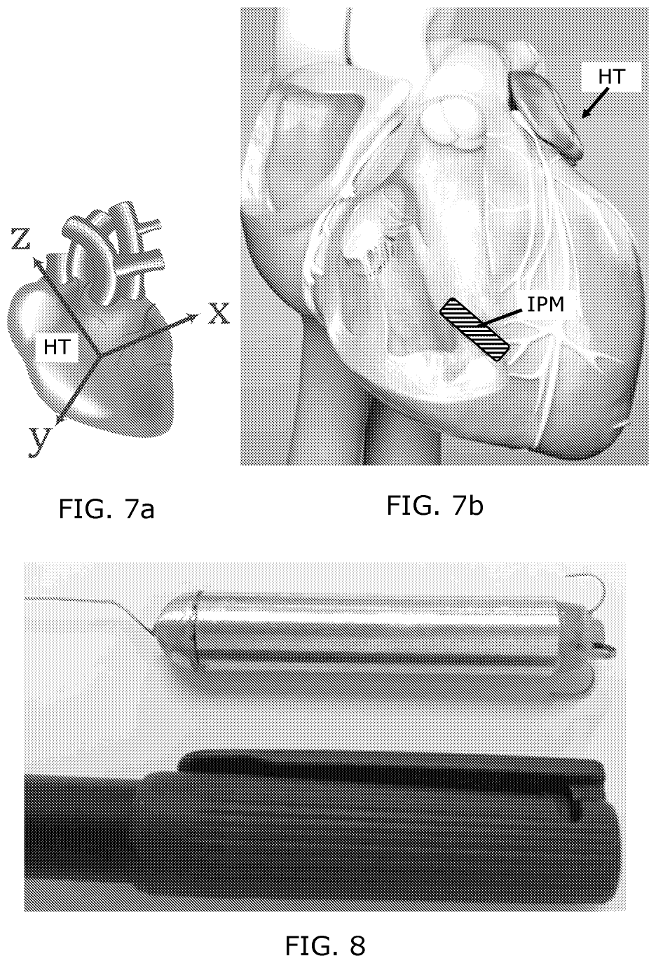

FIG. 7a illustrates a three axis coordinate system X, Y, Z in a sketch of a human or animal heart HT, serving to illustrate that during the heart HT beating, the heart HT performs a complex movement involving movement of many parts of the heart HT in all three directions X, Y, Z. Thus, the heart HT generates a cyclic three dimensional vibratory force when beating.

FIG. 7b illustrates a human heart HT with a leadless intra-cardiac pacemaker IPM with an energy harvester according to the invention within the human or animal heart HT. The pacemaker IPM is positioned inside the heart in the right ventricle and attached to the endocardium of the heart HT as for normal use. With this position in the heart HT, the heart beats generates a vibratory force on of the pacemaker IMP, and based on this, the energy harvester inside it can generate power enough to power all electronic components of the pacemaker IPM.

FIG. 8 illustrates a photo of an intra-cardiac pacemaker prototype with an energy harvesting embodiment of the invention which has been tested in a living pig's heart. The casing has a cylindrical shape with a rounded top and bottom and a length of such as 30 mm and a width of such as 9 mm. In one end of the pacemaker stimulation electrodes and hooks for attachment to the heart tissue is seen, while an electric wire is shown in the opposite end of the pacemaker which was special to the prototype for animal test in order to allow external electric measurements on the energy harvester when positioned in side the pig's heart. A cap of a pen is shown below to illustrate size of the pacemaker.

The tested pacemaker prototype basically corresponds to the embodiment shown in FIG. 6. The prototype had an energy harvester with two cubic compartments each with side lengths of about 5 mm. The impact object in each of the compartments was a ⅛ inch metal sphere with a mass of 0.13 grams as impact object. The two compartments were each formed by one single piezoelectric element bent into the cubic shape. The piezoelectric element was a layered structure of two piezoelectric sheets with in intermediate layer of non-piezoelectric material. One electric output electrode from each compartment was applied to respective AC to DC converters.

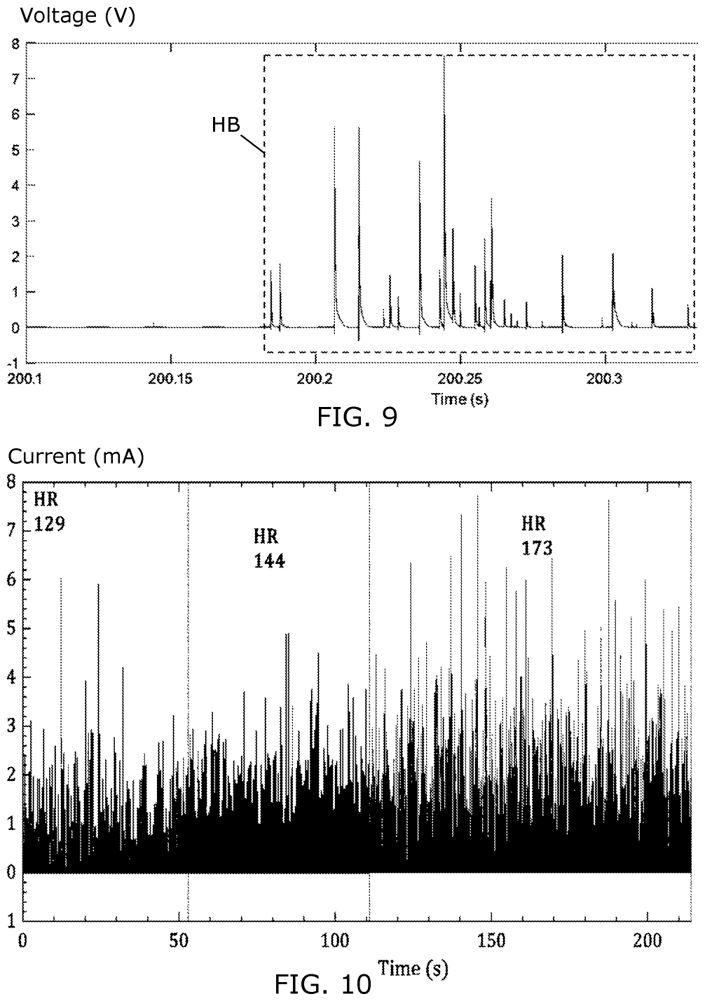

FIG. 9-11 illustrate graphs indicating electrical measurement results of the tested energy harvesting prototype when mounted in the test in a living pig's heart corresponding to the same position in the heart as for normal implanting of a leadless intra-cardiac pacemaker in a human heart. The maximum displacement observed in the test during a heartbeat was approximately 12 mm.

FIG. 9 shows measure voltage from the energy harvester versus time. As seen, the energy harvester generates voltage pulses corresponding to the impact object hitting the piezoelectric walls of the compartment. Pulses of up to 8 V are seen. The dashed box indicates the pulses corresponding to the duration of one single heartbeat HB, and as seen, several significant pulses are observed during one single heartbeat HB, e.g. 4 pulses exceed a magnitude of more than 4 V.

FIG. 10 illustrates short-circuit current versus time, as measured from the energy harvester at three different heart rates HR, namely 129, 144, and 173 beats per minute. As seen, pulses with a magnitude of more than 2 mA are frequently seen, and pulses of more than 5 mA are also seen, especially at the highest heart rate HR.

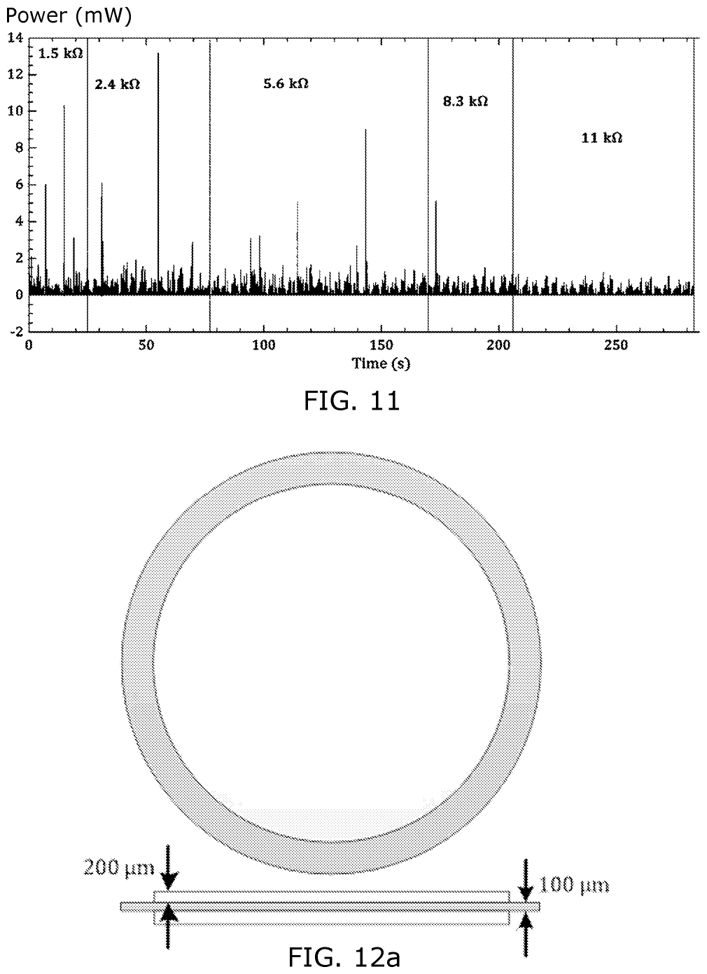

FIG. 11 illustrates calculated electric power versus time based on measured voltage and current from the energy harvester. The calculated power is shown for five different load impedances: 1.5 kΩ, 2.4 kΩ, 5.6 kΩ, 8.3 kΩ, and 11 kΩ.

In general, it has been calculated based on the animal test, that the prototype energy harvester can generate an average power of 16.4 W power by occupying a volume of 0.55 cm3 (diameter 8.4 mm and length 11 mm). The peak optimal power is 2.5 mW, the maximum generated voltage is 12.0 V, and the maximum generated current is 6.0 mA.

An average power of 16.4 μW is sufficient to power the electronics of a leadless intra-cardiac pacemaker. Thus, in preferred intra-cardiac pacemaker embodiments, the size and weight of the required energy storage is very limited and especially an energy storage, such as comprising a rechargeable battery and/or a capacitor, can be used which occupies significantly a smaller volume than the batteries in prior art intra-cardiac pacemakers.

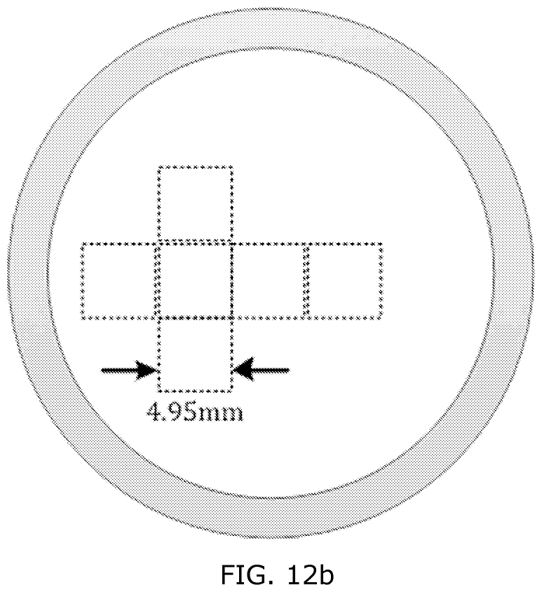

FIG. 12a, 12b illustrate sketches of initial steps of a possible manufacturing of an energy harvesting compartment based on one single piece or sheet of piezoelectric material. In the following, more details of a specific process will be described in relation to FIGS. 12a and 12b, but generally, the idea is to manufacture an energy harvester compartment from a piezoelectric material by providing a suitably shaped piezoelectric element and bending it into the shape of the compartment, such as a cubic shaped compartment. Hereby the compartment is formed by one single piezoelectric element and with all side walls being made of piezoelectric material. With only one single piezoelectric element, only one electric output terminal is required, thereby reducing the complexity following electric circuits involved in the energy harvesting compared to having a compartment with several separate piezoelectric elements.

FIG. 12a shows a piezoelectric double-layer disc shaped sheet seen in top view and in side view, where the two piezoelectric layers (white) are seen in a sandwich with an intermediate layer non-piezoelectric material (grey). The piezoelectric layers are shown here as having a thickness of 200 μm and with an intermediate layer with a thickness of 100 μm. Other thicknesses may of course be used, e.g. resulting in a double sheet having a total thickness of such as 100-2000 μm, such as 100-1000 μm.

FIG. 12b shows the unfolded drawing of a preferred shape of the energy harvester compartment or part thereof provided on the sheet. Specifically, an unfolded cube is illustrated and may be preferred, here specifically with a side length of 4.95 mm. However, a side length of 3-8 mm, such as 4-6 mm may in general be preferred. Likewise, the unfolded shape may be an unfolded rectangular box rather than a cube, if preferred. Still further, the unfolded shape may be a hexagonal sidewall for a compartment.

The unfolded shape on the sheet can be cut e.g. using a laser-cutting process, and the unfolded surface is then folded or bent to form the compartment, with the specific illustration, the result will be a compartment with a cubic shape. The impact object, e.g. a sphere or ball, is placed inside the compartment before completing the folding or bending. Finally, an electric output terminal, e.g. an electric wire or an electric connector, is preferably connected to the piezoelectric material.

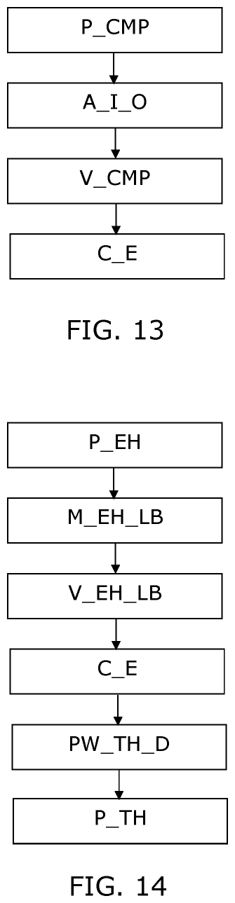

FIG. 13 illustrates steps of an embodiment of a method for harvesting electric energy from an external vibratory force, such as a vibratory force caused by movement of a part of a living human or animal. First steps is providing P_CMP a compartment with one or more wall elements forming a cavity inside, wherein at least one wall element comprises a piezoelectric material. Next, arranging A_I_O an impact object inside the cavity of the compartment. Next, vibrating V_CMP the compartment by the external vibratory force so as to cause the impact object to move inside the cavity and to generate impacts on a surface of the at least one wall element comprising piezoelectric material, and during the vibration of the compartment, collecting C_E electric energy from the piezoelectric material caused by said impacts. The step of collecting may involve converting electric impulses received from the piezoelectric material to a DC voltage. Further, the DC voltage may be applied to an electric storage, and/or applied to an electric energy consuming device.

FIG. 14 illustrate steps of an embodiment of a method of providing a therapy on a living human or animal body, based on electric energy from an energy harvester according to the invention. The method comprises in a first step providing P_EH an energy harvester according to the invention. Next, mounting M_EH_LB the energy harvester on or in a part of the living human or animal body. By mounting is understood covering various ways of fixing or attaching the energy harvester to a part of the living body, including e.g. that the energy harvester can be attached to an outer part of the body, or it could be implanted into a part of the body, such as implanted into the heart. Specifically, the energy harvester may form part of an intra-cardiac pacemaker which is implanted in the human or animal heart. Next, vibrating V_EH_LB the energy harvester by a vibratory force provided by the living human or animal body, e.g. the human or animal heart. By this is understood that movements of the part of the living human or animal body where the energy harvester is positioned is transferred to the compartment of the energy harvester so as to vibrate the compartment, thereby generating electric energy. Next, collecting electric energy C_E from the energy harvester, and powering PW_TH_D a therapy device based on the collected electric energy. Finally, providing a therapy P_TH on the living human or animal body by means of the therapy device. Specifically, this may comprise providing electric stimulation, such as electric stimulation to the human or animal heart.

To sum up, the invention provides an energy harvester for generating electric energy from an external vibratory force, such as a vibratory force caused by movement of a part of a living human or animal. The energy harvester has compartment with wall element(s) forming a cavity inside. At least one wall element of the compartment has a piezoelectric element connected to an electric output terminal. An impact object is positioned inside the cavity of the compartment. The impact object is configured to move inside the cavity and to generate an impact on a surface of the wall element(s), so as to cause the piezoelectric element to generate an electric output on the electric output terminal. The compartment may have a cubic shape, e.g. made of a single piezoelectric element folded into shape, e.g. a two-layer piezoelectric element. The impact object may be a metal or ceramics object with a spherical shape. Especially, the energy harvester may be used to power an implantable medical device, e.g. an intra-cardiac pacemaker. An energy harvester housing within an intra-cardiac pacemaker has been tested to be sufficient for powering the pacemaker stimulation circuit by the vibratory force caused by heartbeats.

Although the present invention has been described in connection with the specified embodiments, it should not be construed as being in any way limited to the presented examples. The scope of the present invention is to be interpreted in the light of the accompanying claim set. In the context of the claims, the terms “including” or “includes” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1-54. (canceled)

55. An energy harvester for generating electric energy from an external vibratory force, the energy harvester comprising:
at least one compartment with one or more wall elements forming a cavity inside, wherein one or more of the wall elements comprises at least one piezoelectric element connected to an electric output terminal, and
at least one impact object positioned inside the cavity in the at least one compartment, wherein the at least one impact object is configured to move inside the cavity and to generate an impact on a surface of the one or more wall elements when the at least one compartment is excited by the external vibratory force, so as to cause the at least one piezoelectric element to generate an electric output on the electric output terminal,
wherein at least one impact object optionally has a spherical shape or a polygonal shape, and
wherein an entire compartment is made of one piezoelectric element bent into a preferred compartment shape.

56. The energy harvester according to claim 55, comprising a compartment wherein the one or more wall elements form a cavity with an inner surface having a curved part.

57. The energy harvester according to claim 56, wherein the one or more wall elements form a cavity with a concave inner surface.

58. The energy harvester according to claim 56, wherein the one or more wall elements form a cavity with an inner surface having a plurality of curved parts.

59. The energy harvester according to claim 55, comprising a compartment wherein the one or more wall elements form a cavity with an inner surface comprising at least one plane part.

60. The energy harvester according to claim 59, wherein the one or more wall elements form a cavity with an inner surface comprising a plurality of plane parts.

61. The energy harvester according to claim 60, wherein the one or more wall elements form a cavity with an inner surface comprising 6 plane parts.

62. The energy harvester according to claim 55, comprising a compartment wherein the one or more wall elements form a cavity with an inner surface comprising a plurality of differently shaped parts.

63. The energy harvester according to claim 55, wherein the one or more wall elements form an enclosure around the at least one impact object, wherein the enclosure has a maximum opening size which is smaller than a size of the at least one impact object.

64. The energy harvester according to claim 55, comprising a plurality of compartments, each compartment comprising an electric output terminal.

65. The energy harvester according to claim 64, wherein the electric output terminals from the plurality of compartments are interconnected to form one common electric output.

66. The energy harvester according to claim 55, wherein the cavity of one of the at least one compartment has a volume of 1-1000 mm³.

67. The energy harvester according to claim 55, wherein one wall element comprising one single piezoelectric element is bent or folded so as to form the compartment, such as a cubic compartment.

68. The energy harvester according to claim 55, wherein the piezoelectric element is a piezoelectric sandwich element which comprises two piezoelectric sheets and an intermediate layer of non-piezoelectric material.

69. The energy harvester according to claim 55, wherein at least one impact object positioned inside one of the at least one compartment comprises a metal or a ceramic.

70. The energy harvester according to claim 55, wherein the at least one impact object has a spherical shape having a diameter of 1-5 mm, the at least one impact object having a mass of 0.05-3 g.

71. The energy harvester according to claim 70, wherein the at least one compartment has a cubic shape with a side length being within 2-10 mm.

72. A device, comprising at least one energy harvester according to claim 55.

73. The device according to claim 72, wherein the device comprises a medical device.

74. A method, comprising effecting operation of a device according to claim 72 so as to provide a therapy to a living human or animal body.
